(19) United States
(12) Patent Application Publication
(10) Pub. No.: US 2019/0029301 A1
(43) Pub. Date: Jan. 31, 2019

(54) USE OF LONG-TERM FASTING MIMICKING AS DIETARY TREATMENT FOR MULTIPLE MYELOMA AND OTHER CANCERS

(71) Applicant: UNIVERSITY OF SOUTHERN CALIFORNIA, Los Angeles, CA (US)

(72) Inventors: Valter D. LONGO, Playa del Rey, CA (US); Woodring E. WRIGHT, Arlington, TX (US)

(73) Assignee: UNIVERSITY OF SOUTHERN CALIFORNIA, Los Angeles, CA (US)

(21) Appl. No.: 16/069,706

(22) PCT Filed: Jan. 12, 2017
(86) PCT No.: PCT/US2017/013158

§ 371 Date: Jul. 12, 2018

Related U.S. Application Data
(60) Provisional application No. 62/277,649, filed on Jan. 12, 2016.

(51) Int. Cl. A23L 33/115; A23L 33/125; A23L 33/17; A61P 35/00; A61K 31/454; A61K 31/69

(52) U.S. Cl. CPC A23L 33/115; A23L 33/125; A23L 33/17; A23V 2002/00; A61K 31/454; A61K 31/69; A61P 35/00

(57) ABSTRACT

A method for treating a subject with multiple myeloma includes a step of identifying a patient having multiple myeloma. A fasting mimicking and enhancing diet is administered to the subject for a predetermined time period of at least 8 days. A diet package for implementing the method is also provided.

### CROSS-REFERENCE TO RELATED APPLICATIONS

This application claims the benefit of U.S. provisional application(s) Ser. No. 62/277,649 filed Jan. 12, 2016, the disclosure of which is incorporated in its entirety by reference herein.

### TECHNICAL FIELD

In at least one aspect, the present invention is related to methods for treating multiple myeloma with a fasting mimicking diet.

### BACKGROUND

Fasting Mimicking and Enhancing Diets (FMED) have been developed to promote the effects of fasting on the sensitization of a variety of cancer cell types to chemotherapy (Differential Stress Sensitization), while protecting normal cells and tissues (Differential Stress Resistance) and avoiding the burden and potential malnourishment associated with fasting. Furthermore, the metabolic conditions produced by the FMED can induce apoptosis independently of chemotherapy in many tumor cell types. Multiple myeloma is routinely inappropriately treated using monotherapy maintenance regimens that foster the development of resistance.

Accordingly, three is a need for improved methods of treating blood cancers such as multiple myeloma.

### SUMMARY

In at least one aspect, the present invention provides a method to treating a subject with multiple myeloma. The method includes a step of identifying a patient having multiple myeloma. A fasting mimicking and enhancing diet is administered to the subject a predetermined time period of at least 8 days.

In another embodiment, a method for reversing drug resistance in a subject having cancer, and in particular, multiple myeloma is provided. The method includes a step of identifying a subject having cancer (e.g., multiple myeloma) and associated drug resistance or at risk of developing drug resistance. A fasting mimicking and enhancing diet is administered to the subject for a predetermined time period. A chemotherapeutic agent for which resistance has developed is administered to the subject prior to and/or concurrently with and/or after administration of the fasting mimicking and enhancing diet.

In another embodiment, a fasting mimicking and enhancing diet package for treating multiple myeloma. The fasting mimicking and enhancing diet package including a first set of rations for a fasting mimicking and enhancing diet to be administered for a predetermined time period to a subject. The fasting mimicking and enhancing diet providing from 4.5 to 7 kilocalories per pound of subject for a first day and 3 to 5 kilocalories per pound of subject per day for a second to final day of the fasting mimicking and enhancing diet. The diet package includes a first portion for the first day that provides less than 30 g of sugar, less than 28 g of proteins, 20 to 30 grams of monounsaturated fats on the first day; between 6 and 10 grams of polyunsaturated fats on the first day; less than 12 g of saturated fats on the first day, and optionally, 12 to 25 grams of glycerol. The diet package also includes a plurality of additional portions, one portion for each of the second day (day 2) to the final day, each portion providing less than 20 g of sugar, less than 18 g of proteins, 10 to15 grams of monounsaturated fats; 3 to 5 grams of polyunsaturated fats; less than 6 grams of saturated fats; and 12 to 25 grams of glycerol. Characteristically, the predetermined time period being at least 8 days such that the final day being a day greater than or equal to day 8.

Embodiments disclosed herein show the effects of inducing a highly evolutionarily conserved fasting response to sensitize multiple myeloma cells to therapy and overcome bortezomib resistance, while protecting normal cells. A second but related objective is to critique the current guidelines for the treatment of myeloma, where the panel members have only provided an un-interpreted catalogue of results. Their recommendations have treated poorly designed studies with the same gravitas as insightful ones. Importantly, this report provides the framework for clinical trials evaluating strategies to avoid development of drug resistance and thereby improve patient outcome, in part by combining standard of care therapies with much broader acting FMEDs which minimize the acquisition of resistance.

### BRIEF DESCRIPTION OF THE DRAWINGS

FIG. 1 provides a plot of the free kappa light chain for a patient treated by the Syn regimen;

FIG. 2 provides a plot of the free kappa light chain for a patient treated by the with an FMED; and

FIG. 3 provides a plot of the free kappa light chain for a patient treated with an FMED and the syn regimen.

### DETAILED DESCRIPTION

Reference will now be made in detail to presently preferred compositions, embodiments, and methods of the present invention which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

Abbreviations:

“MM” means multiple myeloma.

“FMED” means Fasting Mimicking and Enhancing Diet.

The terms “kilocalorie” (kcal) and “Calorie” refer to the food calorie. The term “calorie” refers to the so-called small calorie.

The term “subject” refers to a human or animal, including all mammals such as primates (particularly higher primates), sheep, dog, rodents (e.g., mouse or rat), guinea pig, goat, pig, cat, rabbit, and cow.

The term “fasting mimicking and enhancing diet” means a diet that mimics the effects of fasting typically by providing a subject with at most 50% of their normal caloric intake. The term “fasting mimicking and enhancing diet” means is sometimes simply referred to as a “fasting mimicking diet.” These diets include those diets that have been referred to as fasting mimicking diets. Examples of useful fasting mimicking and enhancing diets and method for monitoring the effects of these diets on markers such as IGF-1 and IGFBP1 in the context of the present invention are set forth in U.S. patent application Ser. No. 14/273,946 filed May 9, 2014; Ser. No. 14/497,752 filed Sep. 26, 2014; Ser. No. 12/910,508 filed Oct. 22, 2010; Ser. No. 13/643,673 filed Oct. 26, 2012; Ser. No. 13/982,307 filed Jul. 29, 2013; Ser. No. 14/060,494 filed Oct. 22, 2013; Ser. No. 14/178,953 filed Feb. 12, 2014; Ser. No. 14/320,996 filed Jul. 1, 2014; Ser. No. 14/671,622 filed Mar. 27, 2015; the entire disclosure of these patent applications is hereby incorporated by reference. The fasting mimicking diet set forth in U.S. patent applications Ser. Nos. 14/060,494 and 14/178,953 are found to be particularly useful in the present invention.

In an embodiment, a method of treating a subject with multiple myeloma is provided. The method includes a step of identifying a patient having multiple myeloma. A fasting mimicking and enhancing diet is than administered to the subject for a predetermined time period. In some variations, the predetermined time period is equal to or greater than, in increasing order of preference, 5, 7, 10, 12, or 15 days. In addition, the predetermined time period is equal to or less than, in increasing order of preference, 35, 30, 25, 22, or 17 days. In a refinement, the predetermined time period is from 5 to 25 days. In another refinement, the predetermined time period is from 8 to 28 days. In still another refinement, the predetermined time period is from 8 to 22 days. In yet another refinement, the predetermined time period is from 10 to 17 days. In a variation, the fasting mimicking and enhancing diet can be administered prior to and/or concurrently with and/or after administration of a chemotherapeutic agent or other cancer drug to the subject. Examples of such cancer drugs include chemotherapy drugs as well as lenalidomide, bortezomib, and combinations thereof.

In another embodiment, a method for reversing or preventing drug resistance in a subject having cancer, and in particular, multiple myeloma is provided. The method includes a step of identifying a subject having cancer (e.g., multiple myeloma) and associated drug resistance. A fasting mimicking and enhancing diet is administered to the subject for a predetermined time period. A chemotherapeutic agent or other cancer drug to which resistance has developed or anticipated is administered to the subject prior to and/or concurrently with and/or after administration of the fasting mimicking and enhancing diet. Examples for the predetermined time period are the same a set forth above. Examples of such chemotherapeutic agents include, but are not limited to, lenalidomide, bortezomib, and combinations thereof.

In some variations of the methods set forth above, the fasting mimicking and enhancing diet is repeated at predetermined intervals. For example, the fasting mimicking and enhancing diet can be initiated once a month for the duration of the subject's treatment which can be 3 months to a year or more (e.g., 1 to 5 years).

In some variations, the methods set forth above alleviate one or more symptoms of multiple myeloma such as bone pain, bone fracture, fatigue, infection, neurological problems while increasing life expectancy (e.g., increasing the chances for survival) and decreasing tumor burden (at least for a while). In particular, the methods herein can reduce free kappa light chains. Therefore, the amount of free kappa light chains can be measured to monitor the subject's response to treatment. In a refinement, the methods can reduce free kappa light chains by at least 40%, 50%, 60% or 70% in a subject having multiple myeloma

In some variations, the fasting mimicking and enhancing diet for each of the methods set forth herein provides at most, in increasing order of preference, 50%, 40%, or 30% of the subject's normal caloric intake. In a refinement, the fasting mimicking diet provides at least, in increasing order of preference, 5%, 10%, or 20% of the subject's normal caloric intake. The subject's normal caloric intake is the number of kcal that the subject consumes to maintain his/her weight. The subject's normal caloric intake may be estimated by interviewing the subject or by consideration of a subject's weight. As a rough guide, subject's normal caloric intake is on average 2600 kcal/day for men and 1850 kcal/day for women. In certain instances, the fasting mimicking diet provides the subject with from 700 to 1200 kcal/day. In a particularly useful refinement, the fasting mimicking diet provides a male subject of average weight with about 1100 kcal/day and a female subject of average weight with 900 kcal/day.

In certain variations, the fasting mimicking and enhancing diet provides from 4.5 to 7 kilocalories per pound of subject for a first day (day 1) and then 3 to 5 kilocalories per pound of subject per day for the second to the final day. After a cycle of the fasting mimicking and enhancing diet, a second diet is administered to the subject for a second time period. In a refinement, the second diet provides an overall calorie consumption that is within 20 percent of a subject's normal calorie consumption for 10 to 26 days (e.g., immediately) following the fasting mimicking and enhancing diet.

In another embodiment, a first diet and diet package for implementing the FMED diet protocol set forth above is provided. The diet of this embodiment is derived from U.S. patent application Ser. No. 14/060,494 which provides a new type of fasting mimicking diet that provides about 900 Kcal; the entire disclosure of which is hereby incorporated by reference. This diet is was much more satisfying than prior diets, and was to be tested to verify that it still induced the fasting response. The diet package includes a first set of rations for a fasting mimicking diet to be administered for a first time period (i.e., the predetermined time period set forth above) to a subject. The fasting mimicking diet provides from 4.5 to 7 kilocalories per pound of subject for a first day and 3 to 5 kilocalories per pound of subject per day for a second to final day of the fasting mimicking diet. The diet package includes rations that provide less than 30 g of sugar on the first day; less than 20 g of sugar on the each of the second day to the final day; less than 28 g of proteins on the first day; less than 18 g of proteins on days the second to fifth days; 20 to 30 grams of monounsaturated fats on the first day; 10 to15 grams of monounsaturated fats on the each of the second day to the final day; between 6 and 10 grams of polyunsaturated fats on the first day; 3 to 5 grams of polyunsaturated fats on the each of the second day to the final day; less than 12 g of saturated fats on the first day; less than 6 grams of saturated fats on the each of the second day to the final day; and 12 to 25 grams of glycerol per day on the each of the second day to the final day. In a refinement, the diet package further includes sufficient rations to provide the micronutrients set forth below. In a further refinement, the diet package provides instructions providing details of the methods set forth above. In particular, the instructions state that a fasting mimicking and enhancing diet implemented by the diet package is to be administered for at least 8 days (or the number of days and manner as set forth herein). Typically, the final day is a day selected from days 8 -25 (i.e., day 8, day 9, day 10, day 11, day 12, day 13, day 14, day 15, day 16, day 17, day 18, day 19, day 20, day 21, day 22, day 23, day 24, or day 25). In a refinement, the final day is a day selected from days 8 -22 (i.e., day 8, day 9, day 10, day 11, day 12, day 13, day 14, day 15, day 16, day 17, day 18, day 19, day 20, day 21, or day 22). Typically, the fats on all days are derived from a combination of the following: Almonds, Macadamia Nuts, Pecans, Coconut, Coconut oil, Olive Oil and Flaxseed. In a refinement, the FMED diet includes over 50% of the recommended daily value of dietary fiber on all days. In the further refinement, the amount of dietary fiber is greater than 15 grams per day on all days of the diet. The diet can also contain 12-25 grams of glycerol per day on each of the second to the final day. In a refinement, glycerol is provided at 0.1 grams per pound body weight/day.

In a variation, the FMED and associated diet package includes the following micronutrients (at least 95% non-animal based): over 5,000 IU of vitamin A per day (day 1 to the final day); 60-240 mg of vitamin C per day (day 1 to the final day); 400-800 mg of Calcium per day (day 1 to the final day); 7.2-14.4 mg of Iron per day (day 1 to the final day); 200-400 mg of Magnesium per day (day 1 to the final day); 1-2 mg of copper per day (day 1 to the final day); 1-2 mg of Manganese per day (day 1 to the final day); 3.5-7 mcg of Selenium per day (day 1 to the final day); 2-4 mg of Vitamin B1 per day (day 1 to the final day); 2-4 mg of Vitamin B2 per day (day 1 to the final day); 20-30 mg of Vitamin B3 per day (day 1 to the final day); 1-1.5 mg of Vitamin B5 per day (day 1 to the final day); 2-4 mg of Vitamin B6 per day (day 1 to the final day); 240-480 mcg of Vitamin B9 per day (day 1 to the final day); 600-1000 IU of Vitamin D per day (day 1 to the final day); 14-30 mg of Vitamin E per day (day 1 to the final day); over 80 mcg of Vitamin K per day (day 1 to the final day); 16-25 mcg Vitamin B12 are provided during the entire 5-day period; 600 mg of Docosahexaenoic acid (DHA, algae-derived) are provided during the entire 5-day period. The FMED diet provides high micronutrient content mostly (i.e., greater than 50 percent by weight) from natural sources including: Kale, Cashews, Yellow Bell Pepper, Onion, Lemon Juice, Yeast, and Turmeric. Mushroom, Carrot, Olive Oil, Beet Juice, Spinach, Tomato, Collard, Nettle, Thyme, Salt, Pepper, Vitamin B12 (Cyanocobalamin), Beets, Butternut Squash, Collard, Tomato, Oregano, Tomato Juice, Orange Juice, Celery, Romaine Lettuce, Spinach, Cumin, Orange Rind, Citric Acid, Nutmeg, Cloves, and combinations thereof. Table 1 provides an example of additional micronutrient supplementation that can be provided in the FMD diet:

TABLE 1
Micronutrient Supplementation

| | Supplement | Formula | Amount | Amount Range | Unit |
|---|---|---|---|---|---|
| Vit A | | | 1250 IU | 900-1600 | IU |
| Vit C | Ascorbic Acid | C₆H₈O₆ | 15.0000 | 10-20 | mg |
| Ca | Calcium Carbonate | CaCO₃ | 80.0000 | 60-100 | mg |
| Fe | Ferrous Fumarate | C₄H₂FeO₄ | 4.5000 | 3-6 | mg |
| Vit D3 | Cholecalciferol | C₂₇H₄₄O | 0.0025 | 0.001-0.005 | mg |
| Vit E | dl-Alpha Tocopheryl | C₂₉H₅₀O₂ | 5.0000 | 3-7 | mg |
| | Acetate | | | | |
| Vit K | Phytonadione | | 0.0200 | 0.1-0.04 | mg |
| Vit B1 | Thiamine Mononitrate | C₁₂H₁₇N₅O₄S | 0.3750 | 0.15-0.5 | mg |
| Vit B2 | Riboflavin E101 | C₁₇H₂₀N₄O₆ | 0.4250 | 0.2-0.6 | mg |
| Vit B3 | Niacinamide | C₆H₆N₂O | 5.0000 | 3-7 | mg |
| Vit B5 | Calcium Pantothenate | C₁₈H₃₂CaN₂O₁₀ | 2.5000 | 1.5-4.0 | mg |
| Vit B6 | Pyridoxine Hydrochloride | C₈H₁₁NO₃•HCl | 0.5000 | 0.3-0.7 | mg |
| Vit B7 | Biotin | C₁₀H₁₆N₂O₃S | 0.0150 | 0.01-0.02 | mg |
| Vit B9 | Folic Acid | C₁₉H₁₉N₇O₆ | 0.1000 | 0.07-0.14 | mg |
| Vit B12 | Cyanocobalamin | C₆₃H₈₈CoN₁₄O₁₄P | 0.0015 | 0.001-0.002 | mg |
| Cr | Chromium Picolinate | Cr(C6H4NO2)3 | 0.0174 | 0.014-0.022 | mg |
| Cu | Cupric Sulfate | CuSO4 | 0.2500 | 0.18-0.32 | mg |
| I | Potassium Iodide | KI | 0.0375 | 0.03-0.045 | mg |
| Mg | Magnesium Oxide | MgO | 26.0000 | 20-32 | mg |
| Mn | Manganese Sulfate | MnSO₄ | 0.5000 | 0.3-0.7 | mg |
| Mo | Sodium Molybdate | Na₂MoO₄ | 0.0188 | 0.014-0.023 | mg |
| Se | Sodium Selenate | Na₂O₄Se | 0.0175 | 0.014-0.023 | mg |
| Zn | Zinc Oxide | ZnO | 3.7500 | 3-5 | mg |

In refinement of the embodiments set forth above, a 8-25-day supply of diet includes: soups/broths, soft drinks, nut bars and supplements. The diet can be administered as follows: 1) on the first day a 1000-1200 kcal diet with high micronutrient nourishment is provided; 2) for the next 8-22 days a daily diet of 650-800 kcal plus a drink containing a glucose substitution carbon source (e.g., glycerol) providing between 60-120 kcal are provided.

Although the first diet (i.e., fasting mimicking and enhancing diet) encompasses virtually any source of fat, sources high in unsaturated fat, including monounsaturated and polyunsaturated fat sources, are particularly useful (e.g., omega-3/6 essential fatty acids). Suitable examples of monounsaturated food sources include, but are not limited to, peanut butter, olives, nuts (e.g., almonds, pecans, pistachios, cashews), avocado, seeds (e.g., sesame), oils (e.g., olive, sesame, peanut, canola), etc. Suitable examples of polyunsaturated food sources include, but are not limited to, walnuts, seeds (e.g., pumpkin, sunflower), flaxseed, fish (e.g., salmon, tuna, mackerel), oils (e.g., safflower, soybean, corn). The first diet also includes a component selected from the group consisting of vegetable extracts, minerals, omega-3/6 essential fatty acids, and combinations thereof. In one refinement, such a vegetable extract provides the equivalent of 5 recommended daily servings of vegetables. Suitable sources for the vegetable extract include, but are not limited to, bokchoy, kale, lettuce, asparagus, carrot, butternut squash, alfalfa, green peas, tomato, cabbage, cauliflower, beets. Suitable sources for the omega-3/6 essential fatty acids include fish such as salmon, tuna, mackerel, bluefish, swordfish, and the like.

In some variations, the diet package includes a second set of rations for a second diet to be administered to the subject for a second time period. The second diet provides an overall calorie consumption that is within 10 percent of a subject's normal calorie consumption. Although the present invention is not significantly limited by the second time period, the second time period can be from 7 days to 6 months or longer. Typically, the second diet can be administered for 25 to 26 days or longer following the fasting mimicking and enhancing diet.

In another variation, the diet used in the method herein follows the following protocol. In particular, subjects with multiple myeloma are provided with a first diet for a first time period, an optional second diet for a second time period, and an optional third diet for a third time period. The first diet can be the fasting mimicking and enhancing diets set forth above. In some refinements, the first diet provides the subject with at most 50% of the subject's normal caloric intake with at least 50% of the kilocalories being derived from fat, preferably monounsaturated fats. The subject's normal caloric intake is the number of kcal that the subject consumes to maintain his/her weight. As set forth above, the subject's normal caloric intake may be estimated by interviewing the subject or by consideration of a subject's weight. As a rough guide, subject's normal caloric intake is on average 2600 kcal/day for men and 1850 kcal/day for women. In certain instances, the first diet provides the subject with from 700 to 1200 kcal/day. In a particularly useful refinement, the first diet provides the male subject of average weight with about 1100 kcal/day and the female subject of average weight with 900 kcal/day. Typically, the first predetermined period of time is from about 1 to 25 days as set forth above. In order to put the level of fat in the first diet in perspective, the U.S. Food and Drug Administration recommends the following nutritional breakdown for a typical 2000 kilocalorie a day diet: 65 gram fat (about 585 kilocalories), 50 grams protein (about 200 kilocalories), 300 grams total carbohydrates (about 1200 kilocalories). Therefore, in one version of the first diet, a majority of the calories from carbohydrates and proteins are eliminated. Although the first diet of the present variation encompasses virtually any source of fat, sources high in unsaturated fat, including monounsaturated and polyunsaturated fat sources, are particularly useful (e.g., omega-3/6 essential fatty acids). Suitable examples of monounsaturated food sources include, but are not limited to, peanut butter, olives, nuts (e.g., almonds, pecans, pistachios, cashews), avocado, seeds (e.g., sesame), oils (e.g., olive, sesame, peanut, canola), etc. Suitable examples of polyunsaturated food sources include, but are not limited to, walnuts, seeds (e.g., pumpkin, sunflower), flaxseed, fish (e.g., salmon, tuna, mackerel), oils (e.g., safflower, soybean, corn). The first diet also includes a component selected from the group consisting of vegetable extracts, minerals, omega-3/6 essential fatty acids, and combinations thereof. In one refinement, such a vegetable extract provides the equivalent of 5 recommended daily servings of vegetables. Suitable sources for the vegetable extract include, but are not limited to, bokchoy, kale, lettuce, asparagus, carrot, butternut squash, alfalfa, green peas, tomato, cabbage, cauliflower, beets. Suitable sources for the omega-3/6 essential fatty acids include fish such as salmon, tuna, mackerel, bluefish, swordfish, and the like.

The second diet of the present variation provides the subject with at most 900 kcal/day. In certain instances, the second diet provides the subject with at most 200 kcal/day. Typically, the second predetermined period of time is from about 2 to 7 days. In certain particular instances, the second predetermined period of time is 3 days. In still another refinement, the second diet includes a component selected from the group consisting of vegetable extracts, minerals, omega-3/6 essential fatty acids, and combinations thereof. In one refinement, such a vegetable extract provides the equivalent of 5 recommended daily servings of vegetable. Suitable sources for the vegetable extract include, but are not limited to, bokchoy, kale, lettuce, asparagus, carrot, butternut squash, alfalfa, green peas, tomato, cabbage, cauliflower, beets. Suitable sources for the omega-3/6 essential fatty acids include fish oils from salmon, tuna, mackerel, bluefish, swordfish, and the like.

In a variation of the present embodiment, the subject is provided with a third diet for a third predetermined period of time. The third diet is to supplement the normal diet of the subject and can be added to the second diets set forth above. Therefore, the third diet may provide an overall calorie consumption that is within 20 percent of a subject's normal calorie consumption as set forth above. The third diet can also include a replenishing composition. Characteristically, the replenishing composition includes essential amino acids, minerals, and essential fats. Advantageously, the third diet will allow the subject to regain the normal weight and maximize strength. Typically, the third predetermined period of time is at least 5 days. The replenishing composition will also optionally include a number of additional components. For example, the replenishing composition may include a vegetable extract. In one refinement, such a vegetable extract provides the equivalent of 5 recommended daily servings of vegetable. Suitable sources for the vegetable extract include, but are not limited to, bokchoy, kale, lettuce, asparagus, carrot, butternut squash, alfalfa, green peas, tomato, cabbage, cauliflower, beets. The replenishing composition may also include omega-3/6 essential fatty acids, and non-essential amino acids. Examples of suitable non-essential amino acids include, but are not limited to, histidine, serine, taurine, tyrosine, cysteine, glutamine, and combinations thereof. The replenishing composition may also include a multi-mineral tablet containing iron, zinc, copper, magnesium, and calcium and may also contain a vitamin B complex including vitamin B12.

As set forth above, the third diet together with the subject's normal diet will allow the subject to regain the normal weight and maximize strength. Typically, the third predetermined period of time is at least 5 days and may continue indefinitely. In certain instances, the third predetermined period of time is from about 4 days to about 14 days. A week is estimated to be nearly optimal for this purpose. The replenishing composition will also optionally include a number of additional components. For example, the replenishing composition may include a vegetable extract. In one refinement, such a vegetable extract provides the equivalent of 5 recommended daily servings of vegetable. Suitable sources for the vegetable extract include, but are not limited to, bokchoy, kale, lettuce, asparagus, carrot, butternut squash, alfalfa, green peas, tomato, cabbage, cauliflower, beets. The replenishing composition may also include omega-3/6 essential fatty acids, and non-essential amino acids. Examples of suitable non-essential amino acids include, but are not limited to, histidine, serine, taurine, tyrosine, cysteine, glutamine, and combinations thereof. Additional details of the third diet are the same as those set forth above.

The following examples illustrate the various embodiments of the present invention. Those skilled in the art will recognize many variations that are within the spirit of the present invention and scope of the claims.

Methods: Blood values of IGF-1 and IGFBP1 were monitored in order to develop an FMED with the following characteristics: 1) able to reduce or greatly reduce the burden of fasting; 2) able to provide adequate nourishment to patients; and 3) able to promote anti-MM effects, as assessed by measuring the production of free light chains.

Results: MM can be a very slowly growing malignancy. Extending the exposure to FMED conditions for 10-17 days was required to produce a MM cytotoxic effect, evidenced by reduced free light chain levels. Although bortezomib achieves responses in MM, development of resistance underlies disease progression. Decreasing the toxicity of bortezomib by subcutaneous administration coupled with protection of normal cells conferred by the FMED induced-stress-resistance pathways allowed for treatment with “toxic” doses of bortezomib (1.7 mg/m²) to restore bortezomib sensitivity, with minimal side-effects. This provides a new treatment for bortezomib resistance.

Interpretation: The broad acting effect of a FMED has the potential to generally enhance the efficacy of many current myeloma therapies.

Results. The patient is a 64 year old man who presented with a collapsed second cervical vertebra in 2006. The diagnosis of myeloma was confirmed with a CT scan showing multiple lytic lesions throughout ribcage, vertebrae and hip, 4% plasma cells by CD138 histochemistry, an initial monoclonal protein level of 2.77 mg/dL, and an initial free kappa light chain of 483 mg/dL. After stabilization of the neck and irradiation, he was maintained on 10 mg revlimid daily during the period between 12/29/06 and 3/22/2010 until his cells developed resistance. For the period indicated above he had no exposure to any other myeloma treatment. Subsequently, four cycles of revlimid+bortezomib+dexamethasone and three cycles of thalidomide+bortezomib+dexamethasone only produced a modest control of his free kappa light chain tumor marker. A drop in this marker from 60 mg/dL to 8 was obtained using a treatment schedule that exploited the synergism between dexamethasone and bortezomib (see below) in December 2010.

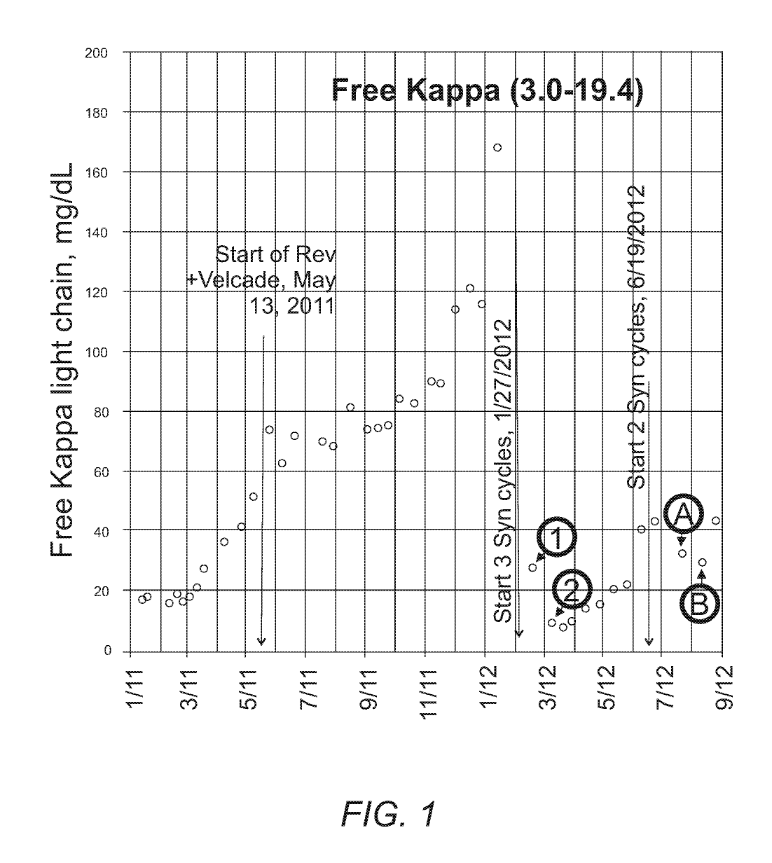

His free kappa began rising in April 2011, and because of persistent steroid side effects, rather than re-treating with all three drugs he opted for a period of maintenance without steroids using just the immunomodulatory agent lenalidomide and the proteosome inhibitor bortezomib. He had previously been on lenalidomide maintenance and as a consequence had developed resistance to lenalidomide, so this lenalidomide+bortezomib treatment amounted to monotherapy with just bortezomib. Rather than staying on it for the shortest possible time while the steroid side effects dissipated, he remained on it and his free kappa started rising in October/November 2011, indicating his cells were now resistant to both lenalidomide and bortezomib. This illustrates the classic development of resistance when monotherapy is used. In January 2012 he began a lenalidomide+bortezomib+dexamethasone regimen that referred to as Syn developed by Ken Anderson that maximizes the synergism between dexamethasone and bortezomib [1](revised to include Biaxin, which competes for the same liver detoxification system used to inactivate dexamethasone and which thus increases the half-life of dexamethasone); the entire disclosure of this publication is hereby incorporated by reference. His cells responded dramatically, with the most sensitive marker of his tumor burden, the free kappa light chain, plummeting from 169 to 27 and 9 (points 1 and 2 in FIG. 1) at the end of the first and second cycle. However, this treatment was close to monotherapy, since his cells were already largely resistant to lenalidomide+bortezomib and the synergism may have simply exacerbated the toxic effect. He remained maintenance-free until his numbers started to rise in May 2012.

Fasting is a highly evolutionarily conserved mechanism for inducing a large number of stress-resistance pathways to protect cells and tissues in times of difficulty. A chronic 30%-40% reduction in the caloric intake below that of ad lib fed animals (calorie restriction, CR) can induce stress responses, but this response is much less effective compared to that caused by complete fasting, evidenced both by sensitization of tumor cells to therapy as well as protection of normal cells. Prolonged fasting is much more effective compared to CR since: 1) it more potently reduces glucose and IGF-I levels while increasing the levels of ketone bodies and IGF-I inhibitor IGFBP1; and 2) it promotes the death of white blood cells, probably in an attempt to minimize energy expenditure. Based upon data showing that a three day water-only (mouse) or 4-5 day water only (human) fast induces the fasting response, a patient undergoing therapy for MM performed cycles of a five-day low calorie, low carbohydrate and low protein Fasting Mimicking and Enhancing Diet (FMED) as set forth in U.S. patent application Ser. No. 14/060,494 filed Oct. 22, 2013 to mimic the effects of fasting on IGF-I and IGFBP1, ketone bodies and glucose. Variations of this 5 day low calorie cycles were carried out approximately every 2 weeks during the period of January 2012 to November 2012 in order to test different components and determine how many calories could be provided with different limits on protein, carbohydrates and fat while still inducing the appropriate fasting response markers.

His cells exhibited resistance to the Syn regimen when it was used again in June 2012, as seen by the minimal drop from the pre-treatment level of 43 to only 32 (point A in FIG. 1) and 29 (point B in FIG. 1) after the first two cycles of treatment. This again illustrates the development of resistant cells when not using multiple effective interventions simultaneously.

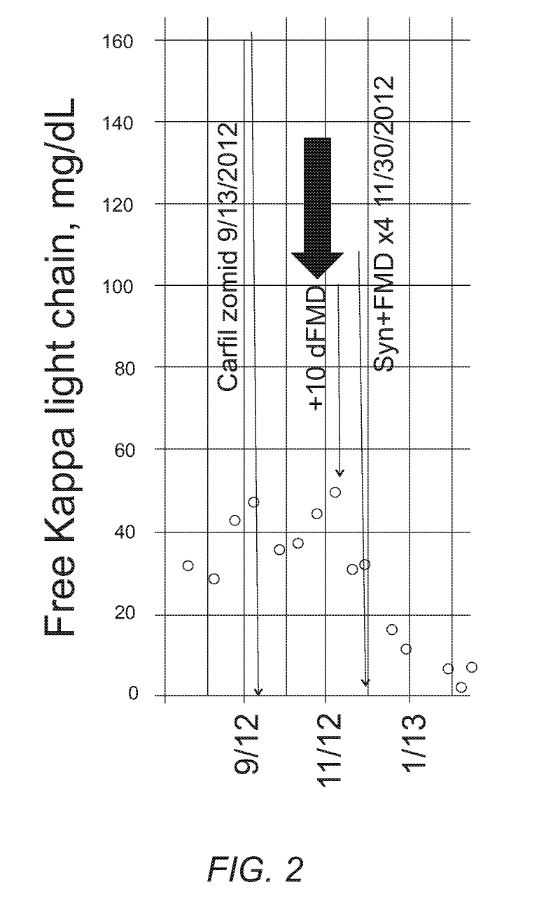

At that time, the patient was undergoing a failing test of replacing bortezomib with carfilzomib (FIG. 2), and it was decided that since multiple myeloma is a very slowly growing tumor, perhaps the period of FMED needed to be significantly extended beyond five days in order to have an effect. Thus, before knowing whether or not the new diet actually did induce the fasting response, a 10-day FMED was tried in order to cover at least two of the carfilzomib treatments. FIG. 2 shows the dramatic response, where his free kappa light chain plummeted. This demonstrates that his tumor cells were sensitive to the conditions produce by the fasting mimicking diet (FMED), since they died rapidly even when being treated with an ineffective drug. Since his cells were at least weakly responsive to the Syn regimen, it was used to replace carfilzomib to consolidate the FMED response.

It has been shown that oncogene mutations prevent tumor cells from responding to fasting [2], and that the metabolic changes produced in the body can cause them to die.

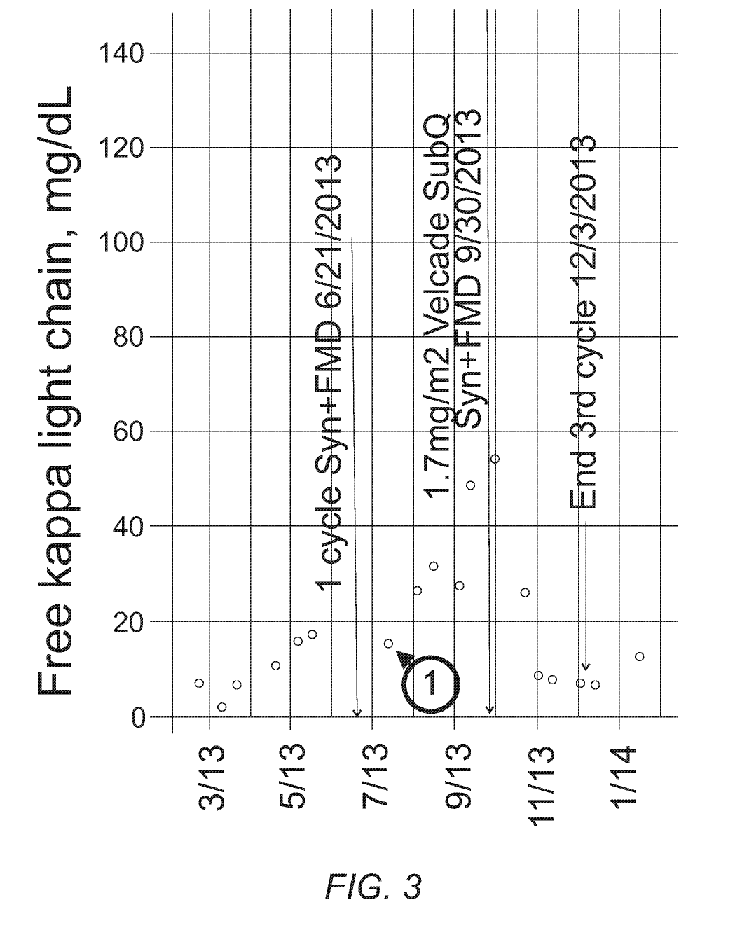

Following the treatments depicted in FIG. 2, the patient went maintenance free. He next received a single cycle of Syn+FMED to maintain the tumor population size as small as possible in order to minimize the probability of developing resistant cells. However, his myeloma now showed only a minimal response (point 1 in FIG. 3), strongly suggesting that his tumor cells had become resistant to the FMED conditions induced by the fasting mimicking diet. One explanation for this failure is the selection of tumor cells resistant to FMED conditions. During the previous 2.5 years, the patient had undergone ˜40-50 five day diets containing numerous variations in content (see above).

SubQ bortezomib has been reported to be as effective as IV bortezomib but with reduced side effects, presumably because of reducing the peak blood levels contributing to toxicity while maintaining the sustained blood levels needed to effect myeloma cells. The patient had previously only used IV bortezomib, since it had been effective up to that point. Resistance to bortezomib is thought to result from overproduction of proteosomal subunits [4], that then require a higher concentration of drug before enough subunits are blocked to reduce the degradation of misfolded myeloma antibodies. The highest recommended dose of bortezomib is 1.3 mg/m², since the next higher dose of 1.7 mg/m² regularly produces grade 3-4 peripheral neuropathy. Even though his tumor cells had likely developed resistance to FMED conditions, his normal cells should still have responded by the induction of a variety of stress-responses. The patient reasoned that reducing the toxicity of bortezomib by using SubQ rather than IV and using the FMED to induce protective stress-resistance pathways in his neural and supporting glial cells might allow the use of “toxic” doses of bortezomib. FIG. 3 shows the results of three cycles of this treatment. The first two cycles were performed to verify whether the approach worked. FIG. 3 shows the rapid drop in tumor markers that resulted from this combination treatment. Since the “toxic” bortezomib dose was essentially monotherapy, a different IMID was added for a third cycle in the hope that the patient's MM cells would be sensitive and one would be treating with at least two drugs. One third of patients resistant to lenalidomide respond to pomalidomide, which was thus added during the third cycle. The patient's peripheral neuropathy showed only minor progression during these three cycles of therapy.

The patient is now maintenance-free and following his tumor burden. Once his light chains rise to 20-40 mg/dL he will do a single cycle of SubQ “toxic” bortezomib+pomalidomide+FMED, since it is no longer necessary to determine whether or not it works. It is hoped that by the time he needs to re-treat, an additional agent will be available so that a real multi-drug combinations can be used to avoid the development of resistance that consistently occurs when monotherapy is used.

It is important to note that large numbers of myeloma patients are currently bortezomib resistant. Although using just the FMED might be sufficient to kill the cells, we believe that combining approaches is the best way to prevent the development of resistance. If others with myeloma induce the fasting response using the same FMED as the patient, then a single cycle of SubQ bortezomib at 1.7 mg/m² combined with an FMED should result in a dramatic response. Based upon the results of FIG. 2, in which shorter FMED were effective, we are initiating a clinical trial that will use a 13 day FMED to test whether or not other myeloma patients induce the same DR response and whether their cells exhibit the same sensitivity to DR conditions as initially shown for this patient's cells. A 13-day FMD will cover at least two treatments, regardless of whether the patients are on a once/week treatment schedule or a 1,4,8,11 schedule.

Discussion. The above described treatment protocol has great potential to change the treatment paradigm for multiple myeloma but also a variety of other cancers. Treatment would begin with a diagnostic period, in which the goal is not to produce a tumor response but rather to determine sensitivities and/or unexpected intolerance to particular agents (i.e. bortezomib, lenalidomide, and dex). This would be followed by induction cycles using standard doses of a synergistic regimen (i.e., 1.0 rising to 1.3 mg/m² subQ bortezomib d1,4,8,11; 20 mg dexamethasone d1,2,4,5,8,9,11,12; 500 mg biaxin d1-14; 25 mg lenalidomide d1-14)+/−FMED to achieve maximal response with the goal of a minimal residual disease state. At that point, stem cells could be harvested for a potential future autologous transplant, but under a revised protocol that avoided any exposure to clastogenic drugs that would seed the surviving myeloma cells/myeloma stem cell with additional mutations. Thus GM/CSF without accompanying cyclophosphamide treatment could be used to mobilize hematopoietic stem cells. Following stem cell collection, patients would not receive maintenance therapy, but rather periodically be re-treated as indicated by routine follow-up myeloma studies.

There are now large numbers of precedents that establish that multidrug combination therapy is required to control highly mutagenic conditions. These range from the experience demonstrating the necessity of multidrug therapy in HIV and tuberculosis to traditional combination therapies for Hodgkin's disease, lymphoma, and childhood ALL. In spite of these clear demonstrations, review panels continue to legitimize monotherapy maintenance regimens. As just one of many examples, the National Comprehensive Cancer Network Guidelines Version 2. 2014 for Multiple Myeloma contains an entire section on maintenance therapy. Studies have shown that maintenance regimens prolong progression-free survival, but these inevitably lead to the development of resistance. The present report suggests the utility of intermittent combination therapy, coupled with the induction of the dietary stress response, as a strategy to prevent and treat drug resistance and thereby prolong overall survival in MM.

Summary. FMED extended over multiple treatments can effectively kill myeloma cells. FMED can induce protective stress-resistance pathways in normal neural and/or neural supportive cells so that “toxic” doses of bortezomib can be tolerated and used to overcame bortezomib resistance.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Richardson, P. G., et al., Lenalidomide, bortezomib, and dexamethasone combination therapy in patients with newly diagnosed multiple myeloma. Blood, 2010. 116(5): p. 679-86.
2. Lee, C., Safie, F. M.,Raffaghello, L., Wei, M., Madia, F., Parrella, E., Hwang, D., Cohen, P. Bianchi, G., Longo, V.d., Reduced levelsof IGF-1 mediate differential protection of normal and cancer cells in response to fasting and improve chemotherapeutic index. Cancer Research, 2010. 70.
3. Leprivier, G., et al., The eEF2 kinase confers resistance to nutrient deprivation by blocking translation elongation. Cell, 2013. 153(5): p. 1064-79.
4. Lu, S. and J. Wang, The resistance mechanisms of proteasome inhibitor bortezomib. Biomarker Research, 2013. 1(1): p. 13.

What is claimed is:

1. A method for treating a subject with multiple myeloma comprising:
identifying a patient having multiple myeloma; and
administering a fasting mimicking and enhancing diet to the subject a predetermined time period of at least 8 days.

2. The method of claim 1 wherein the fasting mimicking diet is administered for 8 to 22 days.

3. The method of claim 1 wherein the fasting mimicking and enhancing diet provides from 4.5 to 7 kilocalories per pound of subject for a first day and 3 to 5 kilocalories per pound of subject per day for a second day to a final day.

4. The method claim 3 where the fasting mimicking and enhancing diet provides for the first day less than 30 g of sugar, less than 28 g of proteins, 20 to 30 grams of monounsaturated fats on the first day; between 6 and 10 grams of polyunsaturated fats on the first day; less than 12 g of saturated fats on the first day, and optionally, 12 to 25 grams of glycerol; and
provides for each day from the second day to the final day less than 20 g of sugar, less than 18 g of proteins, 10 to15 grams of monounsaturated fats; 3 to 5 grams of polyunsaturated fats; less than 6 grams of saturated fats; and 12 to 25 grams of glycerol.

5. The method of claim 1 further comprising administering a chemotherapeutic agent to the subject.

6. The method of claim 5 the chemotherapeutic agent is selected from the group consisting of lenalidomide, bortezomib, and combinations thereof.

7. A method for revising drug resistance in a subject having multiple myeloma comprising:
identifying a patient having multiple myeloma and chemotherapeutic drug resistance; and
administering a fasting mimicking and enhancing diet to the subject for a predetermined time period of at least 8 days; and
administering a chemotherapeutic agent to the subject.

8. The method of claim 7 wherein the fasting mimicking diet is administered for 8 to 22 days.

9. The method of claim 7 wherein the fasting mimicking and enhancing diet provides from 4.5 to 7 kilocalories per pound of subject for a first day and 3 to 5 kilocalories per pound of subject per day for a second day to a final day.

10. The method claim 9 where the fasting mimicking and enhancing diet provides for the first day less than 30 g of sugar, less than 28 g of proteins, 20 to 30 grams of monounsaturated fats on the first day; between 6 and 10 grams of polyunsaturated fats on the first day; less than 12 g of saturated fats on the first day, and optionally, 12 to 25 grams of glycerol; and
provides for each day from the second day to the final day less than 20 g of sugar, less than 18 g of proteins, 10 to15 grams of monounsaturated fats; 3 to 5 grams of polyunsaturated fats; less than 6 grams of saturated fats; and 12 to 25 grams of glycerol.

11. The method of claim 7 the chemotherapeutic agent is selected from the group consisting of lenalidomide, bortezomib, and combinations thereof.

12. The method of claim 7 the chemotherapeutic agent is lenalidomide.

13. The method of claim 7 wherein the chemotherapeutic agent is bortezomib.

14. A diet package comprising:
a first set of rations for a fasting mimicking and enhancing diet to be administered for a predetermined time period to a subject, the fasting mimicking and enhancing diet to providing from 4.5 to 7 kilocalories per pound of subject for a first day and 3 to 5 kilocalories per pound of subject per day for a second day to a final day of the fasting mimicking and enhancing diet.

15. The diet package of claim 14 including:
a first portion for the first day that provides less than 30 g of sugar, less than 28 g of proteins, 20 to 30 grams of monounsaturated fats on the first day; between 6 and 10 grams of polyunsaturated fats on the first day; less than 12 g of saturated fats on the first day, and optionally, 12 to 25 grams of glycerol; and
a plurality of additional portions, one portion for each of the second day to the final day, each portion providing less than 20 g of sugar, less than 18 g of proteins, 10 to15 grams of monounsaturated fats; 3 to 5 grams of polyunsaturated fats; less than 6 grams of saturated fats; and 12 to 25 grams of glycerol, the predetermined time period being at least 8 days such that the final day being a day greater than or equal to day 8.

16. The diet package of claim 14 wherein the predetermined time period is 8 to 22 days.

17. The diet package of claim 14 wherein the predetermined time period is 10 to 17 days.

18. The diet package of claim 14 further comprising a second set of rations for a second diet to be administered to the subject for a second time period, the second diet providing an overall calorie consumption that is within 10 percent of a subject's normal calorie consumption.

19. The diet package of claim 18 wherein the second diet is administered for 25 to 26 days following the fasting mimicking and enhancing diet.

20. The diet package of claim 14 further comprising instructions that a fasting mimicking and enhancing diet implemented by the diet package is to be administered for at least 8 days.
